(19) United States
(12) Patent Application Publication
(10) Pub. No.: US 2021/0260241 A1
(43) Pub. Date: Aug. 26, 2021

(54) CHITIN/GRAPHENE COMPOSITE SPONGE AND PREPARATION METHOD AND USE THEREOF

(71) Applicant: SOUTH CHINA UNIVERSITY OF TECHNOLOGY, Guangzhou, Guangdong (CN)

(72) Inventors: Xiaoying WANG, Guangdong (CN); Xiaoyun LI, Guangdong (CN); Shanshan LI, Guangdong (CN)

(73) Assignee: South China University of Technology, Guangzhou, Guangdong (CN)

(21) Appl. No.: 17/053,795

(22) PCT Filed: Oct. 31, 2019
(86) PCT No.: PCT/CN2019/114539

§ 371 Date: Nov. 9, 2020

(30) Foreign Application Priority Data
Jan. 31, 2019 (CN) 201910100643.6

(51) Int. Cl. A61L 24/00; C08K 3/04; C08J 9/28; C08J 3/075

(52) U.S. Cl. CPC A61L 24/0078; C08K 3/042; C08J 2305/08; C08J 3/075; A61L 2400/04; C08J 9/286

(57) ABSTRACT

Disclosed are a chitin/graphene composite sponge and a preparation method and a use thereof. The method comprises mixing and ball-milling a certain amount of flake graphite and chitin, dissolving a mixture of the flake graphite and the chitin in a NaOH/urea solvent, performing centrifugal separation, dispersing evenly, cross-linking with an epichlorohydrin cross-linking agent, standing, dialyzing, and freeze-drying, thus obtaining the chitin/graphene composite sponge.

### BACKGROUND

### Technical Field

The present disclosure belongs to the field of functional materials, and more particularly, relates to a chitin/graphene composite hemostatic sponge and a preparation method thereof, which can be used in related fields such as medical treatment, sanitation, and the like.

### Description of Related Art

At present, a non-absorbable and non-degradable hemostatic material needs to be taken out by a secondary operation after use, and there are risks of blood clot rupture and pulling injury. However, an absorbable and degradable hemostatic material (commonly used hyaluronic acid gel, collagen sponge, and the like) has a good biodegradability, but a raw material used has the problems of a high cost, a complicated purification technology, and the like. Therefore, the development of a degradable hemostatic material with a low-cost and easy-to-prepare raw material has a broad application prospect.

Chitin is cheap in price, rich in sources and simple in extraction technology. Meanwhile, a chitin material with excellent biocompatibility and biodegradability has been proved to have a good swelling property and be used as a hemostatic material. However, the pure chitin material only causes blood cell aggregation through swelling adsorption, thus inducing blood clotting, without meeting a requirement of rapid hemostasis.

At present, most hemostatic materials are added with a functional material to improve the hemostatic effect, and the commonly used functional material comprises adsorptive clay, graphene, mesoporous bioactive glass, and the like. The functional material has a large specific surface area and abundant porous structures, thus being able to adsorb plasma or activate platelets to promote the blood clotting. The clay generates heat during hemostasis, and has a problem of leakage, thus limiting its commercial application in hemostasis. Graphene has a relatively large specific surface area, and is easy to interact with blood to promote the blood clotting.

However, a commonly used preparation method of graphene has the problems of a lengthy reaction process, a toxic reagent used, harm to the environment, a high cost, a difficulty in large-scale production, and the like.

### SUMMARY

### Technical problem

### Solution of problem

### Technical solution

The present disclosure aims to provide a biodegradable chitin/graphene hemostatic sponge which is green in preparation and low in cost and a preparation method thereof.

According to the present disclosure, a swelling ability of chitin and adsorption and procoagulant ability of graphene are combined, and flake graphite and chitin are mixed and exfoliated to obtain a chitin/graphene composite material, which is prepared into a composite hemostatic sponge.

The present disclosure is specifically realized by the following technical solutions.

Flake graphite and chitin are mixed and ball-milled to obtain a chitin/graphene composite material, which is dissolved in a NaOH/urea solvent, centrifugally separated, cross-linked with a cross-linking agent epichlorohydrin, stood, dialyzed, and freeze-dried, thus obtaining a chitin/graphene composite hemostatic sponge.

Further, a preparation method of the chitin/graphene composite sponge specifically comprises the following steps:

(1) preparation of the chitin/graphene composite material: mixing the flake graphite and the chitin, drying the mixture of the flake graphite and the chitin in a vacuum oven at 80° C. to 105° C. for 4 hours to 8 hours, and then ball-milling for 4 hours to 8 hours;

(2) preparation of chitin/graphene composite hydrogel: dissolving the composite material obtained in step (1) in a NaOH/urea solvent by a freeze-thawing method, removing unexfoliated graphite through centrifugation, dropwisely adding the cross-linking agent epichlorohydrin into the above solution, stirring at 0° C. to 4° C. for 0.2 hour to 2 hours to obtain an even solution, and then standing at 0° C. to 4° C. for 8 hours to 24 hours, thus obtaining the chitin/graphene hydrogel; and

(3) preparation of the chitin/graphene composite hemostatic sponge: dialyzing the chitin/graphene hydrogel obtained in step (2) at 40° C. to 60° C. for 7 days to 14 days, and freeze-drying at −40° C. to −80° C. for 48 hours to 72 hours.

Preferably, a mass ratio of the chitin to the flake graphite in step (1) is 200:1 to 20:1.

Preferably, a mass ratio of the composite material to the solvent in step (2) is 1:100 to 6:100

Preferably, a condition for the ball-milling in step (1) is an intermittent operation, which is stopped for 10 minutes to 15 minutes every 20 minutes to 30 minutes, and is operated at a rotating speed of 180 r/min to 210 r/min.

Preferably, according to the freeze-thawing method, the composite material is frozen at −20° C. to −40° C., thawed while being stirred at 20° C. to 30° C., and repeated for 2 times to 6 times to evenly disperse the composite material in the solvent.

Preferably, mass fractions of NaOH and urea in the NaOH/urea solvent are 8% to 15% and 2% to 8%, respectively.

Preferably, the centrifugation with a low speed is performed at a rotating speed of 1800 rpm to 3000 rpm for 5 minutes to 20 minutes.

Preferably, an amount of the cross-linking agent required for every 10 g of solution is 0.5 mL to 2 mL.

The chitin/graphene composite sponge is used in a degradable wound hemostatic material, a blood clotting index of the chitin/graphene composite sponge is 9.6 to 18.8, and the chitin/graphene composite sponge is degraded by more than 80% within 6 hours to 12 hours.

Beneficial effect of the present disclosure

Beneficial effect

Compared with the prior art, the present disclosure has the following advantages:

(1) graphene materials are able to be produced in a green manner on a large scale by a low-cost micromechanical exfoliation method, so that the preparation method of the present disclosure is green, low-cost and simple;

(2) the sponge prepared by the present disclosure has an excellent hemostatic effect by combining the swelling ability of the chitin and the adsorption and procoagulant ability of the graphene; and

(3) the sponge prepared by the present disclosure is biodegradable, thus greatly reducing a risk of secondary bleeding resulting from the removal of the hemostatic material.

### BRIEF DESCRIPTION OF THE DRAWINGS

Brief Description of the Drawings

FIG. 1 is a graph of blood clotting indexes of different composite hemostatic sponges.

FIG. 2 is a graph of in vitro degradation over time of different composite hemostatic sponges.

### DESCRIPTION OF THE EMBODIMENTS

Embodiments of the present disclosure

The present disclosure is further described hereinafter with reference to the specific embodiments, but the present disclosure is not limited to these embodiments.

### Embodiment 1

Mixed powder of chitin and flake graphite with a mass ratio of 200:1 was dried in a vacuum oven at 105° C. for 4 hours. Then, the mixed powder was ball-milled in an intermittent operation manner (which was stopped for 10 minutes every 20 minutes, and was operated at a rotating speed of 180 r/min) for 8 hours to obtain a composite material.

The obtained composite material was added into 10% NaOH/4% urea solvent at a mass ratio of 2:100, frozen at −40° C., thawed while being stirred at 23° C., and frozen and thawed twice to evenly disperse the mixture, and unexfoliated graphite was removed through low-speed centrifugation (at a rotating speed of 1800 rpm for 20 minutes). 1 mL of a cross-linking agent epichlorohydrin was dropwisely added into every 10 g of solution, and the mixture was stirred at 0° C. for 0.2 hour to obtain an even solution. The solution was continuously stood at 2° C. for 12 hours to obtain chitin/graphene hydrogel.

The above hydrogel was dialyzed under heating at 50° C. for 10 days, and then freeze-dried at −40° C. for 48 hours, thus obtaining a chitin/graphene composite sponge.

A blood clotting index of the prepared composite hemostatic sponge was 16.1±2.7, and a degradation rate was more than 80% after degradation for 6 hours.

### Embodiment 2

Mixed powder of chitin and flake graphite with a mass ratio of 100:1 was dried in a vacuum oven at 100° C. for 6 hours. Then, the mixed powder was ball-milled in an intermittent operation manner (which was stopped for 12 minutes every 25 minutes, and was operated at a rotating speed of 200 r/min) for 4 hours to obtain a composite material.

The obtained composite material was added into 8% NaOH/2% urea solvent at a mass ratio of 1:100, frozen at −30° C., thawed while being stirred at 30° C., and frozen and thawed for four times to evenly disperse the mixture, and unexfoliated graphite was removed through low-speed centrifugation (at a rotating speed of 2000 rpm for 18 minutes). 0.5 mL of a cross-linking agent epichlorohydrin was dropwisely added into every 10 g of solution, and the mixture was stirred at 4° C. for 1.2 hours to obtain an even solution. The solution was continuously stood at 4° C. for 8 hours to obtain chitin/graphene hydrogel.

The above hydrogel was dialyzed under heating at 40° C. for 14 days, and then freeze-dried at −80° C. for 52 hours, thus obtaining a chitin/graphene composite sponge.

A blood clotting index of the prepared composite hemostatic sponge was 11.2±1.6, and a degradation rate was more than 80% after degradation for 8 hours.

### Embodiment 3

Mixed powder of chitin and flake graphite with a mass ratio of 20:1 was dried in a vacuum oven at 80° C. for 8 hours. Then, the mixed powder was ball-milled in an intermittent operation manner (which was stopped for 15 minutes every 30 minutes, and was operated at a rotating speed of 210 r/min) for 5 hours to obtain a composite material.

The obtained composite material was added into 15% NaOH/8% urea solvent at a mass ratio of 6:100, frozen at −20° C., thawed while being stirred at 20° C., and frozen and thawed for six times to evenly disperse the mixture, and unexfoliated graphite was removed through low-speed centrifugation (at a rotating speed of 3000 rpm for 5 minutes). 2 mL of a cross-linking agent epichlorohydrin was dropwisely added into every 10 g of solution, and the mixture was stirred at 0° C. for 2 hours to obtain an even solution. The solution was continuously stood at 0° C. for 24 hours to obtain chitin/graphene hydrogel.

The above hydrogel was dialyzed under heating at 60 ° C. for 7 days, and then freeze-dried at −50° C. for 72 hours, thus obtaining a chitin/graphene composite sponge.

A blood clotting index of the prepared composite hemostatic sponge was 16.0±2.1, and a degradation rate was more than 80% after degradation for 12 hours.

Evaluation of Chitin/Graphene Sponge

1. Blood clotting index (BCI): 0.05 g of the chitin/graphene sponge was weighed, then 10 μL of a coagulant CaCl₂ (0.2 M) solution was rapidly mixed with 0.1 mL of sodium citrated rabbit blood, and then the mixture was added on the sponge. After oscillation (30 rpm) and incubation at 37° C. for 5 minutes, 12.5 mL of normal saline solution was added to dissolve non-clotted blood. An absorbance value of a hemoglobin solution at 542 nm was determined by UV-vis spectrophometer. A blank control group was set during testing. The blood clotting index was calculated through an absorbance ratio: BCI %=A₁/A₀×100%.

1. Degradation rate: 0.05 g of the chitin/graphene sponge was weighed, then immersed in 10 mL of PBS, and added with 10 mg of lysozyme. The mixture was incubated at 37° C., and transferred to a dialysis bag at a certain time point. A weight loss percentage at each time interval was measured to obtain the degradation rate.

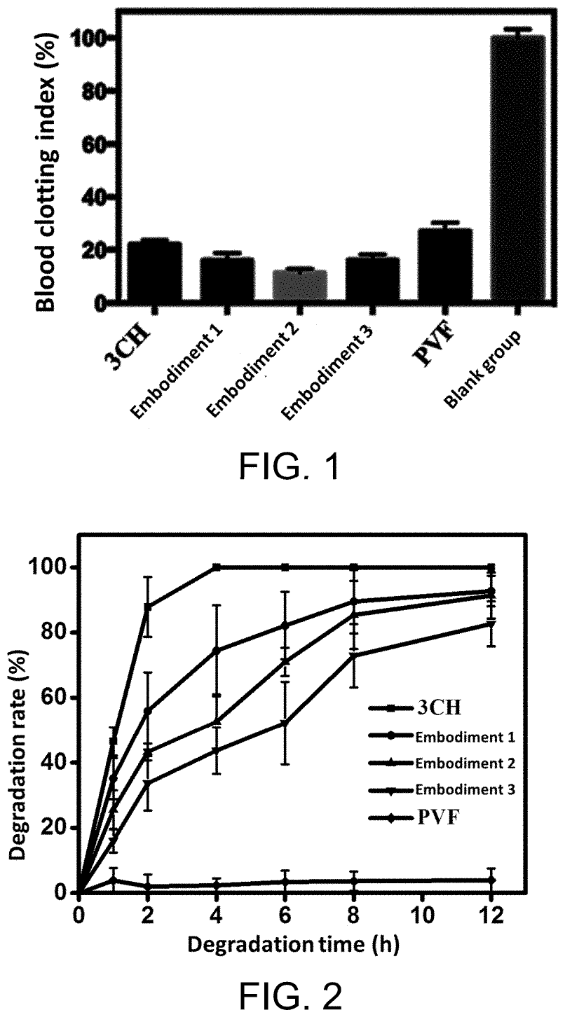

FIG. 1 shows blood clotting indexes of the sponges in the Embodiment 1 to the Embodiment 3, a PVF® and a blank control group. A 3CH is a pure chitin hemostatic sponge with 3% solid content, which means that graphene is not contained. The PVF® is a commercial medical polyvinyl alcohol sponge. Comparison shows that the blood clotting index of the PVF® is 27.0±4.7, and the blood clotting index of the chitin hemostatic sponge is 23.0±1.5. Thus, it can be seen that the chitin hemostatic sponge itself has a better blood clotting effect. The blood clotting index of the hemostatic sponge after being added with the graphene is better than that of the pure chitin hemostatic sponge, and when the mass ratio of the chitin to the flake graphite is 100:1, the blood clotting index is significantly decreased to 11.2. Therefore, the hemostatic sponge prepared by the present disclosure has an excellent hemostatic effect.

In order to measure a blood clotting ability of the sponge, a coagulant of 10 μL of CaCl₂ (0.2 M) solution was rapidly mixed with 0.1 mL of sodium citrated rabbit blood, and then the mixture was added on the sponge. After oscillation (30 rpm) and incubation at 37° C. for 5 minutes, 12.5 mL of normal saline solution was added to dissolve non-clotted blood. An absorbance value of a hemoglobin solution at 542 nm was determined by UV-vis spectrophometer. A blank control group was set during testing. The blood clotting index (BCI) was calculated through the absorbance ratio:

BCI %=A₁/A₀×100%,

wherein A₁ was an absorption value after being treated with the hemostatic sponge, and A₀ was an absorption value of the blank group. The smaller the BCI value was, the better the hemostatic property of the material was. FIG. 2 shows degradation time of the sponges prepared in the Embodiment 1 to the Embodiment 3 and the PVF ®. It can be seen from FIG. 2 that the composite hemostatic sponges with different graphene contents prepared in the present disclosure are almost degraded by more than 80% within 12 hours, while the PVF® is degraded by less than 10% after 12 hours, thus proving that the composite hemostatic sponge of the present disclosure has an excellent biodegradability. An in vitro degradation test was performed under a simulated physiological condition. 50 mg of sample was immersed in 10 mL of PBS, and added with 10 mg of lysozyme. The mixture was incubated at 37° C., and transferred to a dialysis bag at a certain time point. A weight loss percentage at each time interval was measured.

The above embodiments are only the preferred embodiments of the present disclosure, which are only used to explain the present disclosure, and are not intended to limit the present disclosure. The changes, substitutions, modifications, and the like made by those skilled in the art without departing from the spirit of the present disclosure should belong to the scope of protection of the present disclosure.

1. A preparation method of a chitin/graphene composite sponge, wherein the method comprises mixing and ball-milling a flake graphite and a chitin to obtain a chitin/graphene composite material first; and evenly dispersing the chitin/graphene composite material in a solvent, removing unexfoliated graphite through centrifugation, cross-linking with an epichlorohydrin, dialyzing, and freeze-drying, thus obtaining the chitin/graphene composite sponge.

2. The preparation method according to claim 1, wherein the method specifically comprises the following steps:
step 1 preparation of the chitin/graphene composite material: mixing the flake graphite and the chitin, drying the mixture of the flake graphite and the chitin in a vacuum oven at 80° C. to 105° C. for 4 hours to 8 hours, and then ball-milling for 4 hours to 8 hours;
step 2 preparation of a chitin/graphene composite hydrogel: dissolving the composite material obtained in step 1 in a NaOH/urea solvent by a freeze-thawing method, removing the unexfoliated graphite through a centrifugation, dropwisely adding the epichlorohydrin used as a cross-linking agent into the NaOH/urea solvent upon the centrifugation, stirring at 0° C. to 4° C. for 0.2 hour to 2 hours to obtain an even solution, and then standing at 0° C. to 4° C. for 8 hours to 24 hours, thus obtaining the chitin/graphene hydrogel; and
step 3 preparation of the chitin/graphene composite hemostatic sponge: dialyzing the chitin/graphene hydrogel obtained in step 2 at 40° C. to 60° C. for 7 days to 14 days, and freeze-drying at −40° C. to −80° C. for 48 hours to 72 hours.

3. The preparation method according to claim 2, wherein a mass ratio of the chitin to the flake graphite in step 1 is 200:1 to 20:1, and a mass ratio of the composite material to the solvent in step 2 is 1:100 to 6:100.

4. The preparation method according to claim 2, wherein a condition for the ball-milling in step 1 is an intermittent operation, which is stopped for 10 minutes to 15 minutes every 20 minutes to 30 minutes, and is operated at a rotating speed of 180 r/min to 210 r/min.

5. The preparation method according to claim 2, wherein according to the freeze-thawing method, the composite material is frozen at −20° C. to −40° C., thawed while being stirred at 20° C. to 30° C., and repeated for 2 times to 6 times to evenly disperse the composite material in the solvent.

6. The preparation method according to claim 2, wherein mass fractions of a NaOH and an urea in the NaOH/urea solvent are 8% to 15% and 2% to 8%, respectively.

7. The preparation method according to claim 2, wherein the centrifugation is performed at a rotating speed of 1800 rpm to 3000 rpm for 5 minutes to 20 minutes.

8. The preparation method according to claim 2, wherein in step 2, an amount of the cross-linking agent required for every 10 g of solution is 0.5 mL to 2 mL.

9. A chitin/graphene composite sponge prepared by the preparation method according to claim 1.

10. A use of the chitin/graphene composite sponge according to claim 9 in a degradable wound hemostatic material, wherein a blood clotting index of the chitin/graphene composite sponge is 9.6 to 18.8, and the chitin/graphene composite sponge is degraded by more than 80% within 6 hours to 12 hours.

11. A chitin/graphene composite sponge prepared by the preparation method according to claim 2.

12. A chitin/graphene composite sponge prepared by the preparation method according to claim 3.

13. A chitin/graphene composite sponge prepared by the preparation method according to claim 4.

14. A chitin/graphene composite sponge prepared by the preparation method according to claim 5.

15. A chitin/graphene composite sponge prepared by the preparation method according to claim 6.

16. A chitin/graphene composite sponge prepared by the preparation method according to claim 7.

17. A chitin/graphene composite sponge prepared by the preparation method according to claim 8.
